(19) United States
(12) Patent Application Publication
(10) Pub. No.: US 2025/0032447 A1
(43) Pub. Date: Jan. 30, 2025

(54) USE OF UROLITHIN IN THE REMISSION PHASE OF A PRECEDING CANCER THERAPY

(71) Applicants: SOCIETE DES PRODUITS NESTLE S.A., Vevey (CH); LUDWIG INSTITUTE FOR CANCER RESEARCH LTD, Zurich (CH)

(72) Inventors: PIERPAOLO GINEFRA, Lausanne (CH); NICOLA VANNINI, La Croix (Lutry) (CH); GEORGE COUKOS, Chexbres (CH)

(21) Appl. No.: 18/836,884

(22) PCT Filed: Feb. 6, 2023
(86) PCT No.: PCT/EP2023/052751

§ 371 Date: Aug. 8, 2024

(30) Foreign Application Priority Data
Feb. 10, 2022 (EP) 22156024.6

(51) Int. Cl. A61K 31/37; A61K 31/706; A61K 31/714; A61K 35/04; A61K 36/48; A61P 35/00

(52) U.S. Cl. CPC A61K 31/37; A61K 31/706; A61K 31/714; A61K 35/04; A61K 36/48; A61P 35/00

(57) ABSTRACT

The current invention is directed to the therapeutic use of a composition comprising a therapeutically effective amount of a Urolithin or a precursor thereof, preferably suitable for use in reducing the risk of the recurrence of cancer or tumor formation in a patient in need thereof in the remission phase of a preceding cancer therapy in said patient. The invention is furthermore related to a corresponding composition, its method of preparation and a method of reducing the reducing the risk of the recurrence of cancer or tumor formation in a patient in need thereof in the remission phase after a preceding cancer therapy in said patient, wherein the method comprises the following steps: a) Providing a composition comprising a therapeutically effective amount of a Urolithin or a precursor thereof, the composition preferably being a composition as defined herein above; b) Administering the composition comprising the therapeutically effective amount of a Urolithin or a precursor thereof to the patient in need thereof, wherein the patient is in the remission phase after a preceding cancer therapy; and c) Optionally monitoring the patient regularly on the potential recurrence of the cancer treated in the preceding cancer therapy, preferably by using cancer specific biomarkers, preferably concurrent to administering the composition according to step b), preferably in predefined intervals.

### FIELD OF THE INVENTION

The current invention is directed to the therapeutic use of a composition comprising a therapeutically effective amount of a Urolithin or a precursor thereof, preferably suitable for use in reducing the risk of the recurrence of cancer or tumor formation in a patient in need thereof in the remission phase of a preceding cancer therapy in said patient. The invention is furthermore related to a corresponding composition, its method of preparation and a method of reducing the risk of the recurrence of cancer or tumor formation in a patient in need thereof in the remission phase after a preceding cancer therapy in said patient, wherein the method comprises the following steps: a) Providing a composition comprising a therapeutically effective amount of a Urolithin or a precursor thereof, the composition preferably being a composition as defined herein above; b) Administering the composition comprising the therapeutically effective amount of a Urolithin or a precursor thereof to the patient in need thereof, wherein the patient is in the remission phase after a preceding cancer therapy; and c) Optionally monitoring the patient regularly on the potential recurrence of the cancer treated in the preceding cancer therapy, preferably by using cancer specific biomarkers, preferably concurrent to administering the composition according to step b), preferably in predefined intervals.

### BACKGROUND OF THE INVENTION

Cancer is a leading cause of death worldwide, accounting for nearly 10 million deaths in 2020 (see e.g. Ferlay J, Ervik M, Lam F, Colombet M, Mery L, Pineros M, et al. Global Cancer Observatory: Cancer Today. Lyon: International Agency for Research on Cancer; 2020 (https://qco.iarc.fr/today, accessed February 2021). The most common in 2020 in terms of new cases of cancer were: breast (2.26 million cases); lung (2.21 million cases); colon and rectum (1.93 million cases); prostate (1.41 million cases); skin (non-melanoma) (1.20 million cases); and stomach (1.09 million cases). The most common causes of cancer death in 2020 were: lung (1.80 million deaths); colon and rectum (935 000 deaths); liver (830 000 deaths); stomach (769 000 deaths); and breast (685 000 deaths) (see e.g. https://www.who.int/news-room/fact-sheets/detail/cancer).

Cancers are commonly treated by using typically radiotherapy, chemotherapy, surgery and/or in the recent years also by cancer immunotherapy. Although chemotherapy has been used over the years quite successfully as a therapeutic intervention against quite many types of cancer and will most likely be also an important treatment in future, the use of chemotherapy is always accompanied by significant toxic side effects. In order to identify suitable compounds, preferably showing a lowered toxic side effect cancer search has therefore also made efforts in applying other compounds, such as phytonutrients and particularly plant polyphenols in such treatments, partially in addition to classical cancer treatments as a supplemental treatment or in a few cases even as sole therapies, e.g. in the field of cancer immune therapy. Polyphenols are phytochemicals that typically occur in foods and which are therefore considered to exhibit less likely significant toxic side effects compared to classical chemotherapeutics. In addition, such polyphenols are usually present in abundant levels in various fruits and vegetables, which renders provision of such compounds less costly. Some recently discovered type of phytochemicals include gut based polyphenol metabolites of ellagitannins, which have been shown to be effective in the treatment of several type of cancers, such as prostate cancer, uterine cancer, hepatocellular carcinoma, colon cancer, bladder cancer, or breast cancer (see Al-Harbi S A, Abdulrahman A O, Zamzami M A and Khan M I (2021), Urolithins: The Gut Based Polyphenol, Metabolites of Ellagitannins in Cancer Prevention, a Review., Front. Nutr. 8:647582, doi: 10.3389/fnut.2021.647582).

While cancer therapy sometimes overly focuses on the early stages and clinical stages of cancer treatment such as initial treatments before surgery and pathological staging, as well as the immediate clinical steps such as chemotherapy, surgery, etc., an important phase of cancer treatment also includes post-therapy stages. In such post-therapy stages the patient is usually monitored on biomarkers to early detect recurring tumors and to allow, in case of need, restarting as early as possible further cancer treatment of the patient in case initial therapy has not been successful or not sufficient. Such a detection of biomarkers is usually carried out for the first five years after an initial cancer treatment to allow a reliable prediction on the potential recurrence of the tumor and the potential end of the preceding cancer therapy.

In this context, however, many of such cancer therapies, which have to be continued after a first round of treatment restart quite late in the post-therapy phase since allowing for an accumulation of a level of tumors again that is high enough to allow a significant diagnosis. However, this also decreases the chances of successfully fighting the cancer finally in the patient by unnecessarily limiting the time window for post-therapy steps.

In view of the above and facing the significant negative impact of cancers on global health there is an increasing need to more efficiently accompany the post-therapy phase of a preceding cancer treatment and to increase the success rate of the preceding cancer treatment also at a later stage of monitoring, preferably also beyond the typical five years of monitoring the patient in the post-therapy or remission phase.

### SUMMARY OF INVENTION

The inventors have surprisingly found that the above captioned need can be satisfied for various cancer types by the provision and use of an inventive composition as well as a method of using such a composition as it is defined herein and in the attached independent claims. Further preferred embodiments are defined in the attached dependent claims and the specification below.

According to a first embodiment the current invention concerns a composition comprising a therapeutically effective amount of a Urolithin or a precursor thereof, preferably suitable for use in reducing the risk of the recurrence of cancer or tumor formation in a patient in need thereof in the remission phase (after completion) of a preceding cancer therapy in said patient. Preferably, the patient is a human or non-human mammal, preferably a human, optionally a human adult, child or infant. More preferably, the patient is an immune-compromised patient.

According to one aspect, the Urolithin or a precursor thereof is selected from the group consisting of a urolithin, ellagic acid, and an ellagitannin, more preferably is selected from Urolithin A, Urolithin B, Urolithin C or Urolithin D, or a combination thereof. Even more preferably, the urolithin is urolithin A, or a combination of Urolithin A with at least one of Urolithin B, Urolithin C or Urolithin D. Most preferably, the urolithin is urolithin A.

According to a further aspect, the urolithin is administered in an amount of about 500 to 2000 mg/day, preferably in an amount of 750 to 1500 mg/day. Administration may occur either in one, two or three or even more doses per day.

Following a further aspect, the administration of the composition comprising a Urolithin starts at day 0 of the remission phase or at a later stage, e.g. about 1, 2, 3, 4, 5, 6, or 7 days after start of the remission phase, or 1-2 or 2-3 weeks after start of the remission phase, e.g. administration starts in a time frame from day 0 (or day 1, 2, 3, 4, 5, 6 or 7) of the remission phase to about 2 months after start of the remission phase, preferably about 1-2 weeks to 2 months after start of the remission phase, and is preferably carried out for a time of about 2 to 12 months, or longer as defined below. Administration in the remission phase is preferably carried out in a continuous manner.

According to one aspect, the preceding cancer therapy of the patient to be treated concerned treatment of a solid tumor or a liquid tumor, preferably a solid tumor, more preferably a tumor selected from a lymphoma, a carcinoma or a sarcoma, even more preferably a breast cancer, a prostate cancer, an uterine cancer, a hepatocellular carcinoma, a pancreatic cancer, a cervical cancer, a colon cancer, a colorectal cancer, a gastric cancer, a bladder cancer, a skin cancer, a lung cancer or a head and neck cancer, most preferably a pancreatic cancer, a colorectal cancer, a skin cancer or a breast cancer.

Following a particular aspect, the preceding cancer therapy is a cancer surgery, a radiation therapy, a chemotherapy, a targeted cancer therapy, an immunotherapy, a hormonal therapy, or a phytochemical based cancer therapy.

According to a further aspect, the preceding cancer therapy involves use of a Urolithin, preferably Urolithin A, or chemotherapy.

In one aspect, the composition is administered enterally or orally, preferably orally.

In one further aspect, the composition is in the form of a pharmaceutical or nutritional composition, optionally in the form of a food product, food supplement, nutraceutical, food for special medical purpose (FSMP), nutritional supplement, dairy-based drink, low-volume liquid supplement or meal replacement beverage, a plant-based beverage, a gummi, or combinations thereof.

In one other aspect, the composition comprises pomegranate extract, a tamarind extract or a mumijo extract, that provides at least a portion of the at least one Urolithin or precursor thereof. Alternatively or in addition the composition comprises the Urolithin in an isolated form. Preferably, the composition comprises the Urolithin in an isolated form.

According to a second embodiment the current invention concerns the therapeutic or second medical use of a composition as defined above, preferably a composition comprising a therapeutically effective amount of a Urolithin or a precursor thereof for use in reducing the risk of the recurrence of cancer or tumor formation in a patient in need thereof in the remission phase (after completion) of a preceding cancer therapy in said patient. All aspects of the first embodiment also apply for the therapeutic or second medical use according to the second embodiment.

According to a third aspect, the current invention concerns also a method for preparing the inventive composition as defined above, preferably a composition comprising a therapeutically effective amount of a Urolithin or a precursor thereof.

Furthermore, according to a fourth aspect, the current invention concerns a method of reducing the risk of the recurrence of cancer or tumor formation in a patient in need thereof in the remission phase after a preceding cancer therapy in said patient, wherein the method comprises the following steps:
a) Providing a composition comprising a therapeutically effective amount of a Urolithin or a precursor thereof, the composition preferably being an inventive composition as defined herein;
b) Administering the composition according to step a) comprising the therapeutically effective amount of a Urolithin or a precursor thereof to the patient in need thereof, wherein the patient is in the remission phase after a preceding cancer therapy;
c) Optionally monitoring the patient regularly on the potential recurrence of the cancer treated in the preceding cancer therapy, preferably by using cancer specific biomarkers, preferably concurrent to administering the composition according to step b), preferably in predefined intervals.

According to one aspect of the inventive method the composition administered is a composition as defined above according to the first embodiment. All embodiments as defined therein for the first aspect also apply to the composition provided and to be administered herein.

According to a further aspect, the patient to be treated in the inventive method in step b) is a human or non-human mammal, preferably a human, optionally a human adult, child or infant.

Preferably, the patient to be treated in the inventive method in step b) is an immune-compromised patient.

According to one aspect, the Urolithin or a precursor thereof provided in the inventive method in step a) is selected from the group consisting of a urolithin, ellagic acid, and an ellagitannin, more preferably is selected from Urolithin A, Urolithin B, Urolithin C or Urolithin D, or a combination thereof. More preferably, the urolithin is urolithin A, or a combination of Urolithin A with at least one of Urolithin B, Urolithin C or Urolithin D. Even more preferably, the urolithin is urolithin A.

According to a further aspect, the urolithin provided with the composition in step a) and to be administered in the inventive method in step b) is present in the composition in an amount of about 500 to 2000 mg/day, preferably in an amount of 750 to 1500 mg/day. Administration may occur either in one, two or three or even more doses per day.

Following a further aspect, the administration of the composition comprising a Urolithin in the inventive method in step b) starts at day 0 of the remission phase or at a later stage, e.g. about 1, 2, 3, 4, 5, 6, or 7 days after start of the remission phase, or 1-2 or 2-3 weeks after start of the remission phase, e.g. administration starts in a time frame from day 0 (or day 1, 2, 3, 4, 5, 6 or 7) of the remission phase to about 2 months after start of the remission phase, preferably about 1-2 weeks to 2 months after start of the remission phase, and is preferably carried out for about 2 to 12 months, or longer, as defined below. Administration in the remission phase is preferably carried out in a continuous manner.

According to one aspect, the preceding cancer therapy of the patient to be treated concerned treatment of a solid tumor or liquid tumor, preferably a solid tumor, more preferably a lymphoma, a carcinoma or a sarcoma, even more preferably a breast cancer, a prostate cancer, an uterine cancer, a hepatocellular carcinoma, a pancreatic cancer, a cervical cancer, a colon cancer, a colorectal cancer, a gastric cancer, a bladder cancer, a skin cancer, a lung cancer, or a head and neck cancer, most preferably a pancreatic cancer, a colorectal cancer, a skin cancer or a breast cancer.

Following a particular aspect, the preceding cancer therapy of a patient as defined in the inventive method is a cancer surgery, a radiation therapy, a chemotherapy, a targeted cancer therapy, an immunotherapy, a hormonal therapy, or a phytochemical based cancer therapy.

According to a further aspect, the preceding cancer therapy of a patient as defined in the inventive method involves use of a Urolithin, preferably Urolithin A, or chemotherapy.

In one aspect, the composition to be administered in the inventive method in step b) is administered enterally or orally, preferably orally.

In one further aspect, the composition provided in step a) or to be administered in step b) of the inventive method is in the form of a pharmaceutical or nutritional composition, optionally in the form of a food product, food supplement, nutraceutical, food for special medical purpose (FSMP), nutritional supplement, dairy-based drink, low-volume liquid supplement or meal replacement beverage, a plant-based beverage, a gummi, or combinations thereof.

In one other aspect, the composition provided in step a) or to be administered in step b) of the inventive method comprises pomegranate extract, a tamarind extract or a mumijo extract, that provides at least a portion of the at least one urolithin or precursor thereof. Alternatively or in addition the composition provided in step a) or to be administered in step b) of the inventive method comprises the Urolithin in an isolated form. Preferably, the composition provided in step a) or to be administered in step b) of the inventive method comprises the Urolithin in an isolated form.

In a further aspect the composition administered in step a) comprises a combination of a urolithin with a NAD+ precursor and/or Vitamin B12, preferably as defined herein above.

The inventors have surprisingly found that the prevention of the recurrence of tumors or tumor formation is highly efficient when administering to a patient that has received a prior cancer therapy a composition comprising a therapeutically effective amount of a Urolithin or a precursor thereof as defined herein. It was particularly surprising to the inventors that such a post-therapy type of intervention was particularly effective when applied in the remission phase not only in patients with a still working immune system but also immunocompromised patients.

Without being bound by theory, the inventors believe that the underlying effect of the current therapy is based on the effect that UroA dietary supplementation allows to hamper tumor growth through the establishment of an enforced adaptive immune response.

The current invention specifically allows addressing the remission phase of patients having completed a previous cancer therapy and beneficially influence its outcome to increase survival rate as well as to support the body's own immune response to fight the potential recurrence of a tumor. The current invention achieves this by an intervention as early as in the start of the remission phase without unduly waiting for the potential positive detection of biomarkers within the long period of usually 5 years of remission after having completed a previous cancer therapy. This also significantly increases the beneficial diagnosis within the long period of remission. The specific advantage of the inventive treatment lies inter alia in an effective restimulation of T-cell function in patients recovering from cancer, particularly when the inventive composition is administered within the remission period as described below.

### DESCRIPTION OF FIGURES

FIG. 1: shows tumor growth analyses and survival of mice fed with UroA enriched diet in a MC38 model.

FIG. 1A shows schematically the administration schedule during analysis. Mice were fed ad libitum with UroA diet (UA diet) for 4 weeks. Colorectal cancer cells MC38 were subcutaneously injected in the right flank of the mice. Mice receiving the control diet (DMSO diet) were treated identically. Mice of both groups were sacrificed at humane endpoint.

FIGS. 1B and 1C show in two separate experiments (termed Exp1 and Exp2), tumor engraftment and growth, which were significantly reduced in mice exposed ad libitum to UroA enriched diet in both Exp1 and Exp2. In Exp1 (FIG. 1B, left: mean values, right: separate data curves), 5 mice received a DMSO diet and showed an average tumor size of 500 mm³ (filled circles), and 5 mice received Urolithin A enriched diet and showed an average tumor size of 50 mm³ (empty circles). In Exp2 (FIG. 1C), from 3 mice receiving the DMSO diet, 2 mice developed tumors bigger than 100 mm³, while from 3 mice receiving Urolithin A enriched diet only one developed tumors bigger than 100 mm³.

FIG. 1D shows the probability of survival. UroA diet (empty circles) improved overall survival (FIG. 1D) compared to control (DMSO diet, filled circles).

FIG. 2: depicts an analysis of tumor re-challenge in a MC38 model. FIG. 2A shows schematically the administration schedule during analysis in said MC38 re-challenge model. Mice from former UroA treatment group, which survived MC38 tumor challenge (see FIG. 1, Exp. 1 and Exp2) were injected after 7 weeks with the same cancer cell line (MC38) in the opposite flank (FIG. 2A). A control cage group was challenged accordingly. The re-challenged survivor mice and the challenged control cage group were kept in conventional food without UroA for one week before tumor injection.

FIG. 2B shows that mice fed with UroA diet (empty circles) as previously described (FIG. 1b) developed smaller tumors (FIG. 3, average tumor size of 100 mm³) upon re-challenge compared to a freshly challenged control (DMSO diet, filled circles, average tumor size of 600 mm³).

FIG. 2C shows probability of survival and that UroA diet (empty circles) improved overall survival upon re-challenge compared to control (DMSO diet, filled circles).

FIG. 3: shows tumor growth analyses of tumor challenged mice fed with UroA enriched diet in a B16 model (see FIG. 3,top: average values, bottom: separate data curves). In this experiment, 5 mice (B57BL/6) received ad libitum DMSO diet, and 5 mice received ad libitum Urolithin A enriched diet for 4 weeks before tumor injection. Compared to control group, mice fed with UroA diet as previously described (FIG. 1b) developed smaller tumors (FIG. 3, top). While mice fed with DMSO diet showed an average tumor size of 180 mm³ Urolithin A fed mice showed an average tumor size of 70 mm³. FIG. 3, top therefore shows improvement of tumor control by the inventive UroA diet in the less immunogenic B16 tumor model.

FIG. 4: shows that Urolithin A supplementation after 5×10⁵ MC38 cells were subcutaneously injected in the right flank of immunodeficient mice (NSG) does not lead to an enhanced adaptive immune response. In both DMSO control mice and Urolithin A fed mice there is no change in tumors growth rate and volume.

FIG. 5: shows the preventive effect of Urolithin A feeding by boosting the immune system. FIG. 5 shows that while mice fed in DMSO group or in DMSO on/off group showed similar tumor volumes, which reached an average tumor size of 350 mm³ and 430 mm³ at day 17, mice in UroA group and Urolithin A on/off group showed a reduced tumor growth and reached an average tumor size of 200 mm³ at day 17. These results indicate that the Urolithin A diet for 4 weeks has a preventive effect and generates a stronger immune response against cancer cells. The effect was maintained upon Urolithin A diet withdrawal, suggesting a possible imprinting mechanism of Urolithin A on the immune system.

### DETAILED DESCRIPTION OF INVENTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. Endpoints are explicitly included in such ranges.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition or dosage unit “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

A “subject” or “individual” or “patient” is preferably a mammal, more preferably a human. Alternatively, a “subject” or “individual” or “patient” may be a pet, such as a dog or a cat.

As used herein, an “effective amount”, preferably a “therapeutically effective amount”, is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein, namely a composition comprising a Urolithin or an urolithin or a pharmaceutically acceptable salt thereof.

The terms “food,” “food product” and “food composition” mean a product or composition that is intended for ingestion by an individual such as a human and provides at least one nutrient to the individual. A “food,” “food product” and “food composition” typically includes at least one of a protein, a lipid, a carbohydrate and optionally includes one or more vitamins and minerals. A “food,” “food product” and “food composition” may furthermore contain acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers and/or vitamins. The optional ingredients can be added in any suitable amount. The compositions of the present disclosure, including the many embodiments described herein, can comprise, consist of, or consist essentially of the elements disclosed herein, as well as any additional or optional ingredients, components, or elements described herein or otherwise useful in a diet.

The term “unit dosage form”, as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

### EMBODIMENTS OF THE INVENTION

The current invention is described in the following in more detail on basis of the attached embodiments. All aspects described in this context of the attached embodiments may be suitably combined with the embodiments either alone or together with other aspects. Moreover, also different embodiments as detailed below and their aspects may be suitably combined with each other.

### Pharmaceutical and Nutritional Compositions

A first embodiment of this invention is directed to a composition comprising a therapeutically effective amount of a Urolithin or a precursor thereof, preferably suitable for use in reducing the risk of the recurrence of cancer or tumor formation in a patient in need thereof in the remission phase after a preceding cancer therapy in said patient.

Further aspects of the first embodiment, which may be combined with the first embodiment, either alone or in combination, are defined in the following.

According to the first embodiment, the inventive composition comprises a Urolithin. Urolithins are metabolites of dietary ellagic acid derivatives, such as ellagitannins, and are produced in the human gut by gut bacteria.

Ellagitannins are a class of antioxidant polyphenols found in several fruits, particularly pomegranate, strawberries, raspberries and walnuts. Although the absorption of ellagitannins is extremely low, they are rapidly metabolised by the gut microbiota of the large intestine into urolithins.

Due to their superior absorption, urolithins are believed to be the bioactive molecules mediating the effects of ellagitannins. To that end, for example, urolithins were previously shown to have anti-proliferative, antioxidant and anti-inflammatory properties.

Example urolithins within the context of the current invention include elagic acid, urolithin A (3,8-dihydroxyurolithin), urolithin B (3-hydroxyurolithin), urolithin C (3,8,9-trihydroxyurolithin), urolithin D (3,4,8,9-tetrahydroxyurolithin), urolithin A glucuronide and urolithin B glucuronide. Elagic acid and Urolithins A, B, C and D have the following structure:

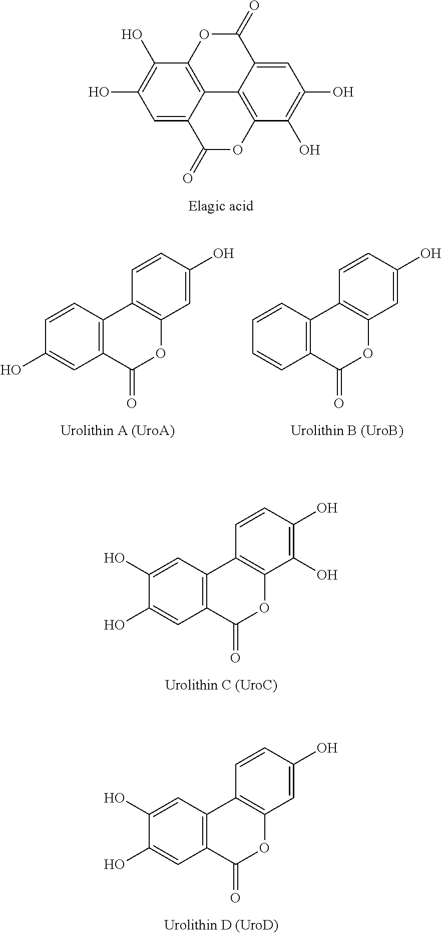 [SMILES: O=c1oc2c(O)c(O)cc3c(=O)oc4c(O)c(O)cc1c4c23 ; O=c1oc2cc(O)ccc2c2ccc(O)cc12 ; O=c1oc2cc(O)ccc2c2ccccc12 ; O=c1oc2c(O)c(O)ccc2c2cc(O)c(O)cc12 ; O=c1oc2cc(O)ccc2c2cc(O)c(O)cc12]

According to a particular aspect of the invention, the Urolithin or the precursor thereof as contained in the inventive composition is selected from the group consisting of a urolithin, ellagic acid, and an ellagitannin, more preferably is selected from Urolithin A, Urolithin B, Urolithin C, Urolithin D, urolithin A glucuronide and urolithin B glucuronide, even more preferably from Urolithin A, Urolithin B, Urolithin C or Urolithin D, or a combination thereof. According to a preferred aspect, the urolithin is urolithin A, or a combination of Urolithin A with at least one of Urolithin B, Urolithin C or Urolithin D, preferably Urolithin B. According to an even more preferred aspect, the urolithin is urolithin A.

The inventive composition may comprise urolithins as defined above, extracted from or comprised in several fruits, such as pomegranate extract, tamarind extract or mumijo extract, strawberries, raspberries and walnuts, that typically provide at least a portion of the at least one urolithin or precursor thereof. Generally, inventive composition may comprise either such a pomegranate extract, tamarind extract or mumijo extract, strawberries, raspberries and walnuts, or more preferably urolithins extracted from such a pomegranate extract, tamarind extract or mumijo extract, strawberries, raspberries and walnuts. Alternatively, or additionally, the inventive composition may comprise any of the urolithins as defined herein in isolated form. An isolated form may be prepared either on basis of natural sources, such as the sources identified above, or may be provided by chemical synthesis.

The urolithin in the inventive composition can be present as a salt or ester, in particular a pharmaceutically-acceptable salt or ester. Pharmaceutically-acceptable salts of the agents of the invention, particularly urolithins, preferably include suitable acid addition or base salts thereof. A review of suitable pharmaceutical salts may be found in Berge et al. (1977) J Pharm Sci 66: 1-19. The invention also includes where appropriate all enantiomers and tautomers of such agents. The skilled person will recognise compounds that possess optical properties (e.g. one or more chiral carbon atoms) or tautomeric characteristics. The corresponding enantiomers and/or tautomers may be isolated/prepared by methods known in the art.

According to one aspect of the invention, the urolithin as contained in the inventive composition may be present in a therapeutically effective amount, preferably in an amount of about 500 to 2000 mg/day, more preferably in an amount of 750 to 1500 mg/day, e.g., an amount of about 500, 600, 700, 750, 800, 900, 1000, 1100, 1200, 1300, 1400, 1500, 1600, 1700, 1800 or 2000 mg/day, or any combination of such values, each value thereby suitable as upper or lower limits, most preferably about 1000 mg/day. The composition may be provided as a single daily dose or in two or more separate daily doses, e.g., 2, 3, 4 or more separate doses per day, preferably either as a single dose or in 2 3, or 4 separate doses per day.

According to one aspect the inventive composition comprising a Urolithin may be provided as an oral or enteral composition, preferably as an oral composition.

According to one other aspect the inventive composition may be in any suitable form, preferably a unit dosage form or any other suitable form. Preferably, such a composition, e.g., as a unit dosage form, is in the form of a pharmaceutical or nutritional composition, optionally in the form of a food product, food supplement or additive, nutraceutical, functional food, food for special medical purpose (FSMP), nutritional supplement, dietary supplement, beverage product, dairy-based drink, low-volume liquid supplement, meal replacement beverage, a plant based beverage, a gummi, or combinations thereof, including milk-powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavored drinks; culinary products; soups; tablets; and/or syrups. In a particularly preferred aspect, the inventive composition comprising a therapeutically effective amount of a urolithin as defined above is in the form of a nutritional supplement, a food additive or a medicament, e.g., a nutraceutical. A food additive or a medicament may be, e.g., in the form of tablets, capsules, pastilles or a liquid for example. Food additives or medicaments are preferably provided as sustained release formulations, allowing a constant supply of the urolithin or precursor thereof for prolonged times. All suitable forms are encompassed herein.

The composition of the invention may further contain protective hydrocolloids (such as gums, proteins, modified starches), binders, film forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Further, the composition may contain an organic or inorganic carrier material suitable for oral or enteral administration as well as vitamins, minerals trace elements and other micronutrients in accordance with the recommendations of government bodies such as the USRDA.

The composition of the invention may contain a protein source, a carbohydrate source and/or a lipid source.

According to one aspect, the inventive composition may therefore optionally comprise at least one protein. The at least one protein can be a dairy based protein, a plant-based protein or an animal-based protein or any combination thereof. Dairy-based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant-based proteins include, for example, soy protein (e.g., all forms including concentrate and isolate), pea protein (e.g., all forms including concentrate and isolate), canola protein (e.g., all forms including concentrate and isolate), other suitable plant proteins that are commercially available are wheat and fractionated wheat proteins, corn and it fractions including zein, rice, oat, potato, peanut, green pea powder, green bean powder, and any proteins derived from beans, lentils, and pulses. Animal-based proteins may be selected from the group consisting of beef, poultry, fish, lamb, seafood, or combinations thereof.

According to a further aspect, the inventive composition may optionally comprise a source of fat. The source of fat includes vegetable fat (such as olive oil, corn oil, sunflower oil, rapeseed oil, hazelnut oil, soy oil, palm oil, coconut oil, canola oil, lecithins, and the like), animal fats (such as milk fat) or any combinations thereof. If the composition of the invention includes a fat source, the fat source preferably provides 5% to 40% of the energy of the formula; for example 20% to 30% of the energy of the formula. In one aspect the inventive composition preferably includes a fatty acid component, which is preferably selected from the group consisting of docosahexaenoic acid (“DHA”), eicosapentaenoic acid (“EPA”), and combinations thereof. DHA and EPA may also be derived from krill, plant sources containing co-3 fatty acids, flaxseed, walnut, algae, and combinations thereof. Certain fatty acids (e.g., 18:4 fatty acids) may also be readily converted to DHA and/or EPA. The inventive composition may also include a-linolenic acid. A suitable fat profile may be obtained using a blend of canola oil, corn oil and high-oleic acid sunflower oil. Alternatively, or additionally, preferably a fish oil or a component thereof may be used.

In a further aspect of the present invention, the inventive composition may optionally comprise fibres or a fibre blend. The fibre blend may contain a mixture of soluble and insoluble fibres. Soluble fibres may include, for example, fructo-oligosaccharides, acacia gum, inulin, etc. Insoluble fibres may include, for example, pea outer fibre.

In a further aspect of the present invention, the inventive composition may optionally comprise a source of carbohydrate. A source of carbohydrates for the composition of the invention may more preferably provide between 40% to 80% of the energy of the composition. The source of carbohydrate includes sucrose, lactose, glucose, fructose, corn syrup solids, maltodextrin, modified starch, amylose starch, tapioca starch, corn starch or any combinations thereof. Inclusion of carbohydrates are advantageous inter alia to allow a simple preparation of the nutritional product, e.g., in form of a dispersion of a powder, etc.

In a further aspect the inventive composition includes an amino acid. The amino acid may preferably be selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof.

In a further aspect, the inventive composition may optionally comprise at least one prebiotic, preferably at least one of the following prebiotics, or any combination thereof: fructo-oligosaccharides, fucosyllactose, galacto-oligosaccharides, galactomannans, gentio-oligosaccharides, gluco-oligosaccharides, guar gum, inulin, isomalto-oligosaccharides, lacto-neotetraose, lacto-sucrose, lactulose, levan, maltodextrins, milk oligosaccharides, partially hydrolyzed guar gum, pectic oligosaccharides, resistant starches, retrograded starch, sialo-oligosaccharides, sialyllactose, soy-oligosaccharides, sugar alcohols, xylo-oligosaccharides, or their hydrolysates, or combinations thereof. The prebiotic is a food substance that selectively promotes the growth of beneficial bacteria or inhibits the growth or mucosal adhesion of pathogenic bacteria in the intestines. The prebiotics are not inactivated in the stomach and/or upper intestine or absorbed in the gastrointestinal tract of the individual ingesting them, but they are fermented by the gastrointestinal microflora and/or by probiotics. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412.

In a further aspect, the inventive composition may optionally comprise at least one probiotic. Probiotics are food-grade microorganisms (alive, including semi-viable or weakened, and/or non-replicating), metabolites, microbial cell preparations or components of microbial cells that could confer health benefits on a host when administered, more specifically probiotics beneficially affect the host by improving intestinal microbial balance, leading to effects on the health or well-being of the host. See, Salminen S, Ouwehand A. Benno Y. et al., Probiotics: how should they be defined?, Trends Food Sci. Technol. 1999:10, 107-10. In general, it is believed that these probiotics inhibit or influence the growth and/or metabolism of pathogenic bacteria in the intestinal tract. The probiotics may also activate the immune function of the host. The probiotic may preferably be selected from the group consisting ofAerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, and combinations thereof.

The inventive composition may optionally comprise a synbiotic. A synbiotic is a supplement that comprises both a prebiotic (at least one of the aforementioned) and a probiotic (at least one of the aforementioned) that work together to improve the microflora of the intestine.

In one additional aspect, the inventive composition includes a further phytonutrient in addition to urolithins. Such a further phytonutrient may preferably be selected from flavonoids, allied phenolic compounds, polyphenolic compounds, terpenoids, alkaloids, sulphur containing compounds, and combinations thereof, and in particular from the group consisting of carotenoids, plant sterols, quercetin, curcumin, limonin, and combinations thereof.

In one further aspect, the inventive composition may optionally comprise minerals. Such mineral(s) may include boron, calcium, chromium, copper, iodine, iron, magnesium, manganese, molybdenum, nickel, phosphorus, potassium, selenium, silicon, tin, vanadium, zinc, or any combinations thereof.

In another aspect, the inventive composition includes an antioxidant. The antioxidant may preferably be selected from the group consisting of astaxanthin, carotenoids, coenzyme Q10 (“CoQ1O”), flavonoids, glutathione, Goji (wolfberry), hesperidin, lacto-wolfberry, lignan, lutein, lycopene, polyphenols, selenium, vitamin A, vitamin C, vitamin E, zeaxanthin, and combinations thereof.

In a further aspect, the inventive composition may comprise a vitamin. The term “vitamin” as used herein, refers to any of various organic substances essential in minute quantities to the nutrition of most animals act especially as coenzymes and precursors of coenzymes in the regulation of metabolic processes. Vitamins have diverse biochemical functions, including function as hormones (for example, vitamin D), antioxidants (for example, vitamin C and vitamin E), and mediators of cell signalling, regulation of cell growth, tissue growth and differentiation (for example, vitamin A). The B complex vitamins, which is the largest in number, function as precursors for enzyme cofactor biomolecules (co-enzymes) that help act as catalysts and substrates in metabolism. The optional ingredients in the inventive composition may therefore also include vitamin(s), such as vitamin A, Vitamin 1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin), preferably in amounts as defined by the FDA for daily recommended intake. Particularly preferred are B vitamins and vitamins E and C, even more preferably vitamin B12.

In one aspect, the inventive composition may comprise additionally to Urolithin a further phytonutrient. The further phytonutrient is at least one of flavonoids, allied phenolic compounds, polyphenolic compounds, terpenoids, alkaloids, sulphur-containing compounds.

Phytonutrients are non-nutritive compounds that are found in many foods. Phytonutrients are functional foods that have health benefits beyond basic nutrition, and are health promoting compounds that come from plant sources. Phytonutrient refers to any chemical produced by a plant that imparts one or more health benefit on a user. Non-limiting examples of phytonutrients include those that are:
i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins.
ii) terpenes (isoprenoids) which include carotenoids (tetraterpenoids) including carotenes (such as, for example, α-carotene, β-carotene, γ-carotene, 5-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and γ-3, −6, and −9 fatty acids (such as, for example, gamma-linolenic acid); triterpenoid (such as, for example, oleanolic acid, ursolic acid, betulinic acid, moronic acid).
iii) betalains which include Betacyanins (such as: betanin, isobetanin, probetanin, neobetanin); and betaxanthins (non-glycosidic versions) (such as, for example, indicaxanthin, and vulgaxanthin).
iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide.
v) protein inhibitors, which include, for example, protease inhibitors.
vi) other organic acids which include oxalic acid, phytic acid (inositol hexaphosphate); tartaric acid; and anacardic acid.

In one aspect, the inventive composition may comprise further to a Urolithin at least one of proteins, lipids and carbohydrates, fibers, antioxidants, vitamins, amino acids, further phytonutrients, prebiotics and/or probiotics, or any combination thereof. For example, the inventive composition may comprise 15-50 wt % proteins, 10-15 wt % lipids, 25-50 wt % carbohydrates and/or 5-10 wt % fibers of total dry weight of the nutritional composition.

The skilled person will be able to determine appropriate amounts of such further ingredients of an inventive composition as defined above depending on the desired dosage of the agent. Dosages may depend on factors such as the age, size and health status of the subject to whom they are administered, on lifestyle, as well as on genetic heritage. Dosages may be in line with the recommended daily intakes (RDA) developed by organisations such as the Food and Nutrition Board of the National Academy of Sciences.

In a further aspect, the inventive composition comprises a combination of a urolithin with a NAD+ precursor and/or Vitamin B12, i.e. a combination of a urolithin with a NAD+ precursor, a combination of a urolithin with Vitamin B12, or a combination of a urolithin with a NAD+ precursor and Vitamin B12.

The NAD⁺ precursor according to the present invention is preferably selected from the group consisting of Nicotinic Acid (Niacin), Nicotinamide (Niacinamide), Nicotinamide Riboside (NR), Reduced Nicotinamide Riboside (NRH), Beta-Nicotinamide Mononucleotide (NMN), Nicotinic acid mononucleotide, Nicotinic acid riboside, a food extract enriched in at least one of these compounds, e.g., a food extract enriched in Nicotinamide adenine dinucleotide (NAD), and mixtures thereof. As used herein, “nicotinamide riboside” includes L-valine and L-phenylalanine esters of nicotinamide riboside.

In a preferred embodiment, the NAD+ precursor is Nicotinamide riboside.

### Nicotinamide Riboside

Nicotinamide riboside (NR) is a pyridine-nucleoside form of vitamin B3, which is a precursor to nicotinamide adenine dinucleotide (NAD).

Nicotinamide riboside has the structure:

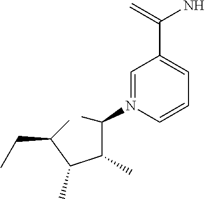

The nicotinamide riboside or NAD+ precursor can be administered in an amount of about 0.001 mg/day to about 2000 mg/day, preferably about 0.001 mg/day to about 1000 mg/day, more preferably about 0.001 mg/day to about 750 mg/day, even more preferably about 0.001 mg/day to about 500 mg/day, most preferably about 0.001 mg/day to about 250 mg/day, for example about 0.001 mg/day to about 100 mg/day, about 0.001 mg/day to about 75 mg/day, about 0.001 mg/day to about 50 mg/day, about 0.001 mg/day to about 25 mg/day, about 0.001 mg/day to about 10 mg/day, or about 0.001 mg/day to about 1 mg/day. Of course, the daily dose can be administered in portions at various hours of the day. However, in any given case, the amount of compound administered will depend on such factors as the solubility of the active component, the formulation used, subject condition (such as weight), and/or the route of administration. For example, the daily doses of nicotinamide riboside disclosed above are non-limiting and, in some embodiments, may be different; in particular, the compositions disclosed herein can be utilized as an acute care food for special medical purposes (FSMP) and contain up to about 2.0 mg nicotinamide riboside/day.

Vitamin B12 (also known as cobalamine) is a class of cobalt-containing hydrosoluble vitamins which cannot be synthesised by the human body and must therefore be acquired from food or synthesised by the gut microbiota.

The vitamin B12 pool in the human body is composed of several forms: cyanocobalamin, which is inactive and requires conversion for activity, and methylcobalamin and adenosylcobalamin, which are the metabolically active forms of vitamin B12.

Two enzymes are known to rely on vitamin B12 as a cofactor: methionine synthase and methylmalonylCoA mutase. Methionine synthase is a cytoplasmic enzyme relying on methyl-cobalamine to convert homocysteine to methionine. It thereby plays a critical role in providing S-adenosylmethionine (SAM) as a methylation donor and preventing the toxic accumulation of homocysteine. Low SAM levels and high homocysteine levels observed upon severe vitamin B12 deficiency impair myelination of peripheral nerves and the spinal cord. Methionine synthase also catalyses the activation of 5-methyl-tetrahydrofolate into the bioactive tetrahydrofolate, which is required for 1-carbon metabolism and DNA synthesis, and thus for efficient red blood cell proliferation. MethylmalonylCoA mutase is a mitochondrial enzyme relying on adenosyl-cobalamine to convert methyl-malonylCoA to succinylCoA, which subsequently enters the TCA cycle. It is implicated in the degradation of branched-chain amino acids and odd-chain length fatty acids, and is essential during embryonic life to control neurological development, but is not vital in adult life

The vitamin B12 of the invention may be in the form of, for example, vitamin B12 itself, the semi-synthetic derivative cyanocobalamin, hydroxocobalamin, methylcobalamin and/or adenosylcobalamin. Methylcobalamin may be particularly effective.

In some embodiments, the vitamin B12 is administered to a subject at 0.1 to 40 times the recommended daily requirement (RDA) of Vitamin B12 per day, e.g. 1 to 10 times the recommended daily requirement (RDA) of Vitamin B12 per day.

The Vitamin B12 may thus be administered in a daily dose of about 10, 20, 30 or 40 times the RDA of the Vitamin B12 per day. Preferably, the daily dose provides 10 to 40, more preferably 10 to 30 or even more preferably 10 to 25 times the RDA of the Vitamin B12 per day, most preferably about 12 to 21 times the RDA of the Vitamin B12 per day.

The United States RDA of Vitamin B12 is 2.4 micrograms daily for humans of age 14 years and older, so such individuals may be administered a daily dose that provides about 0.002 mg to about 0.4 mg of Vitamin B12 per day, preferably 0.02 mg to 0.07 mg of Vitamin B12 per day, more preferably 0.03 mg to 0.05 mg of Vitamin B12 per day.

### Therapeutic Uses

A second embodiment of this invention is directed to the therapeutic use or second medical use of a composition as defined above comprising a therapeutically effective amount of a Urolithin or a precursor thereof, particularly its use in reducing the risk of the recurrence of cancer or tumor formation in a patient in need thereof in the remission phase after a preceding cancer therapy in said patient. The current invention is thereby preferably directed to the prevention of the recurrence of cancer or tumor formation in a patient in need thereof in the remission phase (after completion) of a preceding cancer therapy in said patient. As to whether a preceding cancer therapy in said patient is completed and a remission phase starts, is generally well known to a skilled person and typically decided by the skilled person, e.g., a medical doctor, on basis of clinical criteria or other well-known criteria in this particular field of cancer treatment.

Further aspects of the second embodiment, which may be combined with the inventive therapeutic use applications, either alone or in combination, are defined in the following. Moreover, the second embodiment may be combined with the first embodiment and its aspects as defined above in any combination.

According to one aspect of the invention, the patient as treated in the therapeutic use applications is a human or non-human mammal, preferably a human, optionally a human adult, child or infant. According to a further aspect, the patient is an immune-compromised patient. According to another preferred aspect, the patient is an immuno-compromised human or an immuno-compromised non-human mammal, preferably an immuno-compromised human, optionally an immuno-compromised human adult, child or infant. An infant is in this context typically a human infant, preferably having an age of between 1 to 12 months, a child is typically considered a human having an age of between 12 months and 18 years, human adults are typically considered a human having an age of above 18 years. Alternatively, a patient as treated by the therapeutic use may be a pet, such as a dog or a cat.

According to one aspect of the invention, the urolithin as contained in the inventive composition may be administered in the therapeutic use applications in a therapeutically effective amount, preferably in an amount of about 500 to 2000 mg/day, more preferably in an amount of 750 to 1500 mg/day, e.g., an amount of about 500, 600, 700, 750, 800, 900, 1000, 1100, 1200, 1300, 1400, 1500, 1600, 1700, 1800 or 2000 mg/day, or any combination of such values, each value thereby suitable as upper or lower limits. Administration may occur either via a single daily dose or in two or more separate daily doses, e.g., 2, 3, 4 or more separate doses per day, preferably either as a single dose or in 2, 3, or 4 separate doses per day.

According to another aspect of the invention, the administration of the inventive composition comprising a Urolithin in the therapeutic use applications preferably starts in the remission phase after a preceding cancer therapy in said patient, i.e., after completion of the previous intervention as noted before. Preferably, the administration of the composition comprising a Urolithin starts at day 0 of the remission phase or at a later stage, e.g. about 1, 2, 3, 4, 5, 6, or 7 days after start of the remission phase, or 1-2 or 2-3 weeks after start of the remission phase, e.g. administration starts in a time frame from day 0 (or day 1, 2, 3, 4, 5, 6 or 7) of the remission phase to about 2 months after start of the remission phase, preferably about 1-2 weeks to 2 months, more preferably about 2 weeks to 2 months, more preferably about 3 weeks to 6 weeks, and most preferably about 4-5 weeks or 1 month after start of the remission phase, i.e., after completion of the preceding cancer therapy, in said patient. Thereby, the first day of the remission phase and hence “start of the remission phase” is preferably calculated as “day 0” of the remission phase. Administration in the remission phase is preferably carried out in a continuous manner.

According to a further aspect of the invention, the administration of the composition comprising a Urolithin is preferably carried out in the therapeutic use applications for at least about 1 month, for at least about 2 months, for at least about 2 or about 3 months, preferably for at least about 4 or about 5 months, more preferably at least about 6 or about 7 months in the remission phase, such as about 1 to about 60 months, e.g. about 1 to about 48 months, about 1 to about 36 months, about 1 to about 24 months, or about 1 to about 12 months, preferably such as about 2 to about 60 months, e.g. about 2 to about 48 months, about 2 to about 36 months, about 2 to about 24 months, or about 2 to about 12 months, even more preferably such as about 4 to about 60 months, e.g. about 4 to about 48 months, about 4 to about 36 months, about 4 to about 24 months, or about 4 to about 12 months, etc.

According to a particular aspect of the invention, the administration of the inventive composition comprising a Urolithin preferably starts at day 0 of the remission phase or at a later stage, e.g. about 1, 2, 3, 4, 5, 6, or 7 days after start of the remission phase, or 1-2 or 2-3 weeks after start of the remission phase, e.g. starts in a time frame from day 0 (1, 2, 3, 4, 5, 6 or 7) of the remission phase to about 2 months after start of the remission phase, preferably about 1-2 weeks to 2 months, more preferably about 2 weeks to 2 months, more preferably about 3 weeks to 6 weeks, and most preferably about 4-5 weeks or 1 month after start of the remission phase, i.e., after completion of the preceding cancer therapy, in said patient, and administration of the composition comprising a Urolithin is preferably carried out for at least 1 month, for at least 2 months, for at least about 2 or 3 months, preferably for at least about 4 or 5 months, more preferably for at least about 6 or 7 months in the remission phase, such as about 1 to 60 months, e.g. about 1 to 48 months, about 1 to 36 months, about 1 to 24 months, or about 1 to 12 months, preferably such as for about 2 to 60 months, e.g. 2 to 48 months, 2 to 36 months, 2 to 24 months, or 2 to 12 months, more preferably such as for about 4 to 60 months, e.g. 4 to 48 months, 4 to 36 months, 4 to 24 months, or 4 to 12 months, etc.

According to another aspect of the invention the preceding cancer therapy may be a cancer surgery, a radiation therapy, a chemotherapy, a targeted cancer therapy, an immunotherapy, a hormonal therapy, or a phytochemical based cancer therapy. Such a cancer therapy is preferably directed to one of the cancers as defined before. According to one aspect, the preceding cancer therapy may involve use of a Urolithin as defined herein, preferably Urolithin A, or chemotherapy.

Such a cancer may be, following a further aspect of the invention, a solid tumor or a liquid tumor, preferably a solid tumor, more preferably a lymphoma, a carcinoma or a sarcoma, even more preferably a breast cancer, a prostate cancer, an uterine cancer, a hepatocellular carcinoma, a pancreatic cancer, a cervical cancer, a colon cancer, a colorectal cancer, a gastric cancer, a bladder cancer, a skin cancer, a lung cancer, or a head and neck cancer, most preferably a pancreatic cancer, a colorectal cancer, a skin cancer or a breast cancer. In some aspects, the cancer may also be a haematological cancer, a leukaemia, a lymphoma or a myeloma.

According to one aspect the inventive composition comprising a Urolithin may be administered in the therapeutic use applications orally or enterally, preferably orally. The inventive composition is therefore preferably provided as an oral or enteral composition, preferably as an oral composition.

According to one other aspect the inventive composition to be administered in the therapeutic use applications may be in any suitable form, preferably a unit dosage form or any other suitable form, preferably as defined above.

### Method of Preparation of the Inventive Composition

A third embodiment of the current invention is directed to a method of making a nutritional composition that includes the step of incorporating a therapeutically effective amount of Urolithin as defined herein into a composition as defined above, preferably a pharmaceutical or nutritional composition as defined above. All ingredients as defined for the composition can be added before, parallel or subsequent to the steps of incorporating a therapeutically effective amount of Urolithin as defined herein before. All particular aspects of the herein defined compositions are applicable.

### Method of Treatment

A fourth embodiment of the current invention concerns a method of treatment of a patient in need thereof. The inventive method is directed to reducing the risk of the recurrence of cancer or tumor formation in a patient in need thereof in the remission phase after a preceding cancer therapy in said patient, wherein the method comprises the following steps:
a) Providing a composition comprising a therapeutically effective amount of a Urolithin or a precursor thereof, the composition preferably being a composition as defined herein above;
b) Administering the composition comprising the therapeutically effective amount of a Urolithin or a precursor thereof to the patient in need thereof;
c) Optionally monitoring the patient regularly on the potential recurrence of the cancer treated in the preceding cancer therapy, preferably by using cancer specific biomarkers, preferably concurrent to administering the composition according to step b), preferably in predefined intervals.

The herein described method of treatment aims at preventing the recurrence of cancer or tumor formation in a patient in need thereof in the remission phase (after completion) of a preceding cancer therapy in said patient. As to whether a preceding cancer therapy in said patient is completed and a remission phase starts, is generally well known to a skilled person and typically decided by the skilled person, e.g., a medical doctor, on basis of clinical criteria or other well-known criteria known in this particular field of cancer treatment.

Further aspects of the fourth embodiment, which may be combined with the inventive method either alone or in combination, are defined in the following. Moreover, the fourth embodiment and its aspects may be combined with the first embodiment and any of its aspects as defined above.

According to one aspect, the composition to be administered in the inventive method of treatment in step b) is preferably a composition as defined according to the first embodiment herein. Preferably, such a composition may be in the form of a pharmaceutical or nutritional composition, optionally in the form of a food product, food supplement, nutraceutical, food for special medical purpose (FSMP), nutritional supplement, dairy-based drink, low-volume liquid supplement or meal replacement beverage, a plant-based beverage, a gummi, or combinations thereof.

According to one further aspect, the patient to be treated in the inventive method of treatment is a human or non-human mammal, preferably a human, optionally a human adult, child or infant. According to a further aspect, the patient is an immune-compromised patient. According to another preferred aspect, the patient is an immuno-compromised human or an immuno-compromised non-human mammal, preferably an immuno-compromised human, optionally an immuno-compromised human adult, child or infant. An infant is in this context typically a human infant, preferably having an age of between 1 to 12 months, a child is typically considered to be a human having an age of between 12 months and 18 years, human adults are typically considered to be a human having an age of above 18 years. Alternatively, a patient as treated by the therapeutic use may be a pet, such as a dog or a cat.

According to one other aspect of the invention, the urolithin as defined herein may be administered in step b) of the inventive method of treatment in a therapeutically effective amount, preferably in an amount of about 500 to 2000 mg/day, more preferably in an amount of 750 to 1500 mg/day, e.g., an amount of about 500, 600, 700, 750, 800, 900, 1000, 1100, 1200, 1300, 1400, 1500, 1600, 1700, 1800 or 2000 mg/day, or any combination of such values, each value thereby suitable as upper or lower limits. Administration may occur either via a single daily dose or in two or more separate daily doses, e.g., 2, 3, 4 or more separate doses per day, preferably either as a single dose or in 2 3, or 4 separate doses per day.

The composition to be administered may furthermore comprise further compounds as defined herein, such as vitamins, further phytonutrients, minerals, probiotics, prebiotics, etc. It is noted hereby that a skilled person can readily determine an appropriate dose of such further compounds without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can be of course individual instances where higher or lower dosage ranges are merited, and such are within the scope of the invention.

According to another aspect of the invention, the administration of the inventive composition comprising a Urolithin in step b) of the inventive method of treatment preferably starts after a preceding cancer therapy in said patient, i.e., after completion of the previous intervention as noted before. Preferably, the administration of the composition comprising a Urolithin starts at day 0 of the remission phase or at a later stage, e.g. about 1, 2, 3, 4, 5, 6, or 7 days after start of the remission phase, or about 1-2 or about 2-3 weeks after start of the remission phase, e.g. starts in a time frame from day 0 (or day 1, 2, 3, 4, 5, 6 or 7) of the remission phase to about 2 months after start of the remission phase, preferably about 1-2 weeks to about 2 months, more preferably about 2 weeks to about 2 months, more preferably about 3 weeks to about 6 weeks, and most preferably about 4-5 weeks or about 1 month after start of the remission phase, i.e., after completion of the preceding cancer therapy, in said patient.

According to a further aspect of the invention, the administration of the composition comprising a Urolithin is preferably carried out in step b) of the inventive method of treatment for at least about 1, month, for at least about 2 months, for at least about 2 or about 3 months, preferably for at least about 4 or about 5 months, more preferably for at least about 6 or about 7 months in the remission phase, such as for about 1 to about 60 months, about 1 to about 48 months, about 1 to about 36 months, about 1 to about 24 months, or about 1 to about 12 months, preferably such as for about 2 to about 60 months, e.g. about 2 to about 48 months, about 2 to about 36 months, about 2 to about 24 months, or about 2 to about 12 months, more preferably such as for about 4 to about 60 months, about 4 to about 48 months, about 4 to about 36 months, about 4 to about 24 months, or about 4 to about 12 months, etc.

According to a particular aspect of the invention, the administration of the inventive composition comprising a Urolithin preferably starts in step b) of the inventive method of treatment at day 0 of the remission phase or at a later stage, e.g. about 1, 2, 3, 4, 5, 6, or 7 days after start of the remission phase, or about 1-2 or about 2-3 weeks after start of the remission phase, e.g. starts in a time frame from day 0 (or day 1, 2, 3, 4, 5, 6 or 7) of the remission phase to about 2 months after start of the remission phase, preferably about 1-2 weeks to about 2 months, more preferably about 2 weeks to about 2 months, more preferably about 3 weeks to about 6 weeks, and most preferably about 4-5 weeks or about 1 month after start of the remission phase, i.e., after completion of the preceding cancer therapy, in said patient, and administration of the composition comprising a Urolithin is preferably carried out for at least about 2 or about 3 months, preferably at least about 4 or about 5 months, more preferably at least about 6 or about 7 months in the remission phase, such as about 1 to about 60 months, e.g. about 1 to about 48 months, about 1 to about 36 months, about 1 to about 24 months, or about 1 to about 12 months, preferably such as about 2 to about 60 months, e.g. about 2 to about 48 months, about 2 to about 36 months, about 2 to about 24 months, or about 2 to about 12 months, more preferably such as about 4 to about 60 months, e.g. about 4 to about 48 months, about 4 to about 36 months, about 4 to about 24 months, or about 4 to about 12 months, etc.

According to another aspect of the invention the preceding cancer therapy may be a cancer surgery, a radiation therapy, a chemotherapy, a targeted cancer therapy, an immunotherapy, a hormonal therapy, or a phytochemical based cancer therapy. Such a cancer therapy is preferably directed to one of the cancers as defined before. According to one aspect, the preceding cancer therapy may involve use of a Urolithin as defined herein, preferably Urolithin A, or chemotherapy.

Such a cancer may be, following a further aspect of the invention, a solid tumor or a liquid tumor, preferably a solid tumor, more preferably a lymphoma, a carcinoma or a sarcoma, even more preferably a breast cancer, a prostate cancer, an uterine cancer, a hepatocellular carcinoma, a pancreatic cancer, a cervical cancer, a colon cancer, a colorectal cancer, a gastric cancer, a bladder cancer, a skin cancer, or a head and neck cancer, most preferably a pancreatic cancer, a colorectal cancer, a skin cancer, a lung cancer or a breast cancer. In some aspects, the cancer may also be a haematological cancer, a leukaemia, a lymphoma or a myeloma.

According to one aspect of the inventive method of treatment the inventive composition comprising a Urolithin may be administered orally or enterally, preferably orally. The inventive composition is therefore preferably provided as an oral or enteral composition, preferably as an oral composition. According to one other aspect the inventive composition to be administered in step b) of the inventive method of treatment may be in any suitable form, preferably a unit dosage form or any other suitable form, preferably as defined above.

It is to be appreciated that all references herein to a method of treatment include curative, palliative and prophylactic treatment. The treatment of mammals, particularly humans, is preferred. Both human and veterinary treatments are within the scope of the invention.

Although the composition for use in the invention can be administered alone, they will generally be administered in admixture with a pharmaceutical carrier, excipient or diluent, particularly for human therapy.

### Kit

In another aspect, the present invention also provides a kit containing the inventive composition comprising a therapeutically effective amount of a Urolithin as defined herein. The inventive composition comprising a therapeutically effective amount of a Urolithin may be provided in suitable containers. The kit may also include instructions for use.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Materials and Method

Cell line and in vitro culture: An MC38 colorectal cancer cell line and a B16 cell line were used. The MC38 colorectal cancer cell line (Corbett el al., Can Res, 1975) is a murine colon adenocarcinoma cell line from a C57BL/6 mouse. The cells are adherent and have a fibroblast morphology. MC38 cells will form tumors and metastases post implantation into syngeneic C57BL/6 mice or immunocompromised mice. The B16 melanoma is a murine tumor cell line used for research as a model for human skin cancers. Both MC38 and B16 cancer cell lines are commercially available.

The MC38 colorectal cancer cell line was maintained in RPMI 1640 media (Thermofisher) supplemented with 10% FBS (Gibco), 50 μM beta-mercaptoethanol (Thermofisher), 100 U/ml penicillin/streptomycin (Thermofisher), 1 mM Sodium Pyruvte (Thermofisher).

The B16 cell line were maintained in DMEM+GlutaMAX (Thermofisher) supplemented with 10% FBS (Gibco), and 100 U/ml penicillin/streptomycin (Thermofisher).

### Tumor Engraftment, Diet Treatment and Re-Challenge Experiment:

B57BL/6 mice were purchased from ENVIGO and housed in the animal facility of the University of Lausanne. One week upon arrival mice were fed ad libitum with Urolithin A enriched diet. Urolithin A diet was prepared as follow: 300 mg of Urolithin A (Novalix) was dissolved in DMSO (Applichem) and mixed with 1 kg of “2016 Irradiated Global 16% Rodent diet ground” (ENVIGO) corresponding to a dose of 50 mg/kg/day. For control diet, hereafter named DMSO diet, DMSO (Applichem) was mixed with 1 kg of “2016 Irradiated Global 16% Rodent diet ground” (ENVIGO). The diets were aliquoted in small balls and let them dry under sterile conditions. Mice were fed ad libitum for 4 weeks with DMSO diet or Urolithin A diet before tumor engraftment.

For tumor engraftment, each mouse was injected subcutaneously with 5×10⁵ MC38 cancer cells or 1×10⁵ B16 cancer cells into the right flank.

For the MC38 and B16 experiments 5 mice received ad libitum DMSO diet, and 5 mice received ad libitum Urolithin A enriched diet for 4 weeks before tumor injection. Mice were sacrificed at humane endpoint as established by the Swiss federal regulation and approved by cantonal authorities. Examples of humane endpoint are: tumor size exceeding 1000 m³, loss of weight (15% of the previous time point) and sign of distress. In the MC38 context, 4 out of 5 mice fed with Urolithin A diet did not develop any tumor. These mice were named survivor mice.

For re-challenge experiments, survivor mice were removed from the UroA diet and placed in conventional diet (diet provided by the animal facility of University of Lausanne) for one week. During this week one mouse developed tumor and was excluded from the experiment. The remaining 3 mice were subcutaneously injected with 5×10⁵ MC38 cancer cells. At the same time, another 5 mice (named control cage) were subcutaneously injected with 5×10⁵ MC38 cancer cells. This second tumor injection was done on the left flank of the mouse to discriminate between tumor arising from the first injection from tumor arising from the second, re-challenge, injection.

All experiment were conducted according to the Swiss federal regulations and approved by veterinary authority of Canton Vaud.

Statistical analysis: Data points represent biological replicates and are shown as mean±s.e.m. Statistical significance was determined Student T-tests or Ion-rank (mantel-Cox) test.

### Results

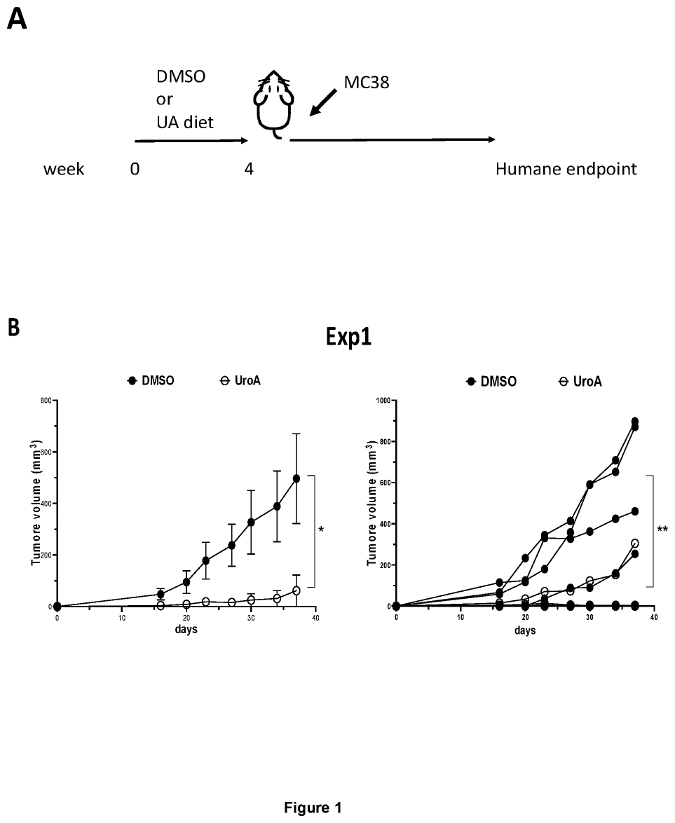

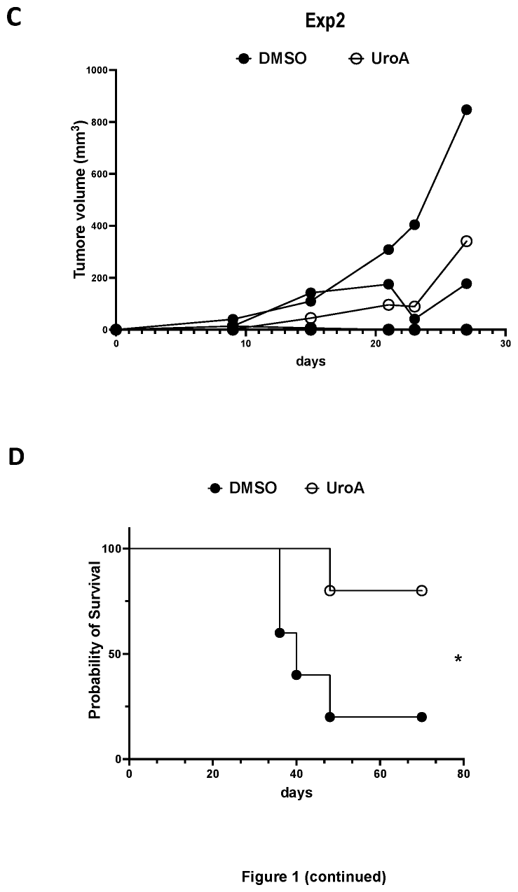

Beside the potential application in in vitro or ex vivo condition, initial tests showed that mice fed with a diet supplemented with UroA increased splenocytes count and overall quality of HSCs. Following this result inventors tested whether UroA enriched diet can improve the ability of the host immune system to control tumor growth. To test this hypothesis, mice were fed ad libitum with UroA diet (see above, Urolithin A diet) and control diet (DMSO, see above) for 4 weeks. Colorectal cancer cells MC38 were subcutaneously injected in the right flank of the mice (FIG. 1A). Tumor volumes were estimated by using the formula: tumor volume=(length×width×width)/2. In two separate experiments (FIG. 1B: Exp1 and FIG. 1B: Exp2), tumors engraftment and growth were significantly reduced in mice exposed ad libitum to UroA enriched diet (FIG. 1B, C).

In Exp1, 5 mice received DMSO diet (see above) and showed an average tumor size of 500 mm³, and 5 mice received Urolithin A enriched diet and showed an average tumor size of 50 mm³. (see FIG. 1B left). FIG. 1B, right shows the single curves (with overlays of UroA curves with one DMSO curve at bottom line).

In Exp2, 3 mice received DMSO diet (see above), 2 out of 3 mice developed tumors bigger than 100 mm³, while out of 3 mice received Urolithin A enriched diet (see above) only one developed tumors bigger than 100 mm³. In addition, UroA diet improved overall survival (FIG. 1D).

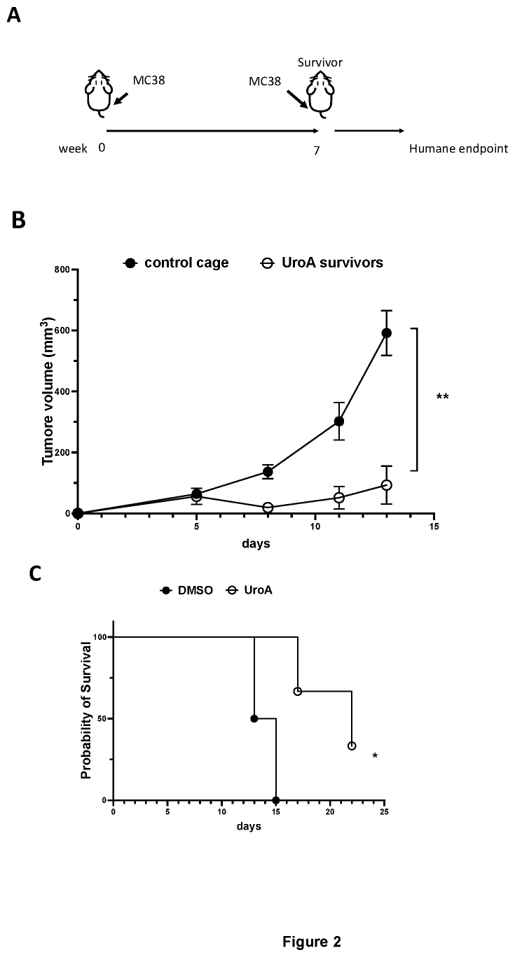

Then, it was tested whether UroA mediated tumor control was exerted through adaptive immune response. It was assumed as a working hypothesis that memory T cells with antitumoral activity would have been generated in the survivor hosts, which would promptly react against a second tumor graft. To test this hypothesis, survivor mice from the UroA group were injected after 7 weeks with the same cancer cell line (MC38) in the opposite flank (FIG. 2A). To exclude any possible contribution of UroA in tumor growth, the mice survivor mice and a control cage group were kept in conventional food without UroA for one week before tumor injection. Notably, survivor mice showed a better tumor control capacity and survival compared to a control group (FIG. 2B and FIG. 2C). While mice fed with DMSO diet showed an average tumor size of 600 mm³ Urolithin A fed mice showed an average tumor size of 100 mm³.

Altogether the current data convincingly demonstrate that UroA dietary supplementation hamper tumor growth through the establishment of an enforced adaptive immunity response.

### Example 2

### Improvement of Tumor Control in a Less Immunogenic Tumor Model

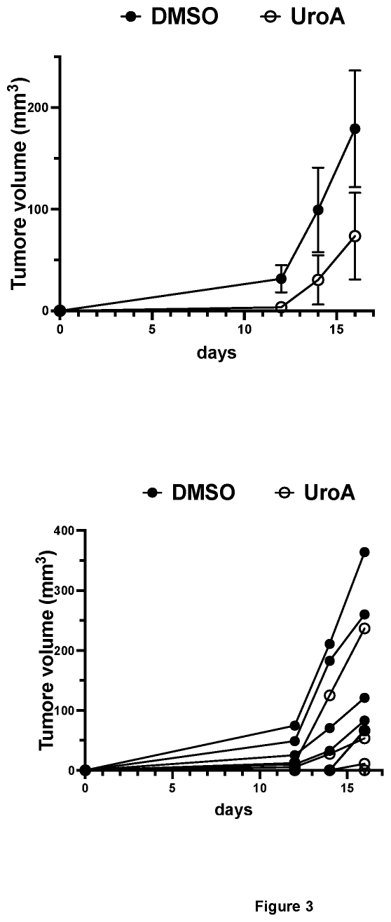

Inventors then also tested whether the inventive UroA diet can improve tumor control in a less immunogenic tumor model such as B16 melanoma (see Lechner, M. G., et al., Immunogenicity of murine solid tumor models as a defining feature of in vivo behavior and response to immunotherapy. J Immunother, 2013. 36(9): p. 477-89). In this experiment, 5 mice (B57BL/6) received ad libitum DMSO diet, and 5 mice received ad libitum Urolithin A enriched diet for 4 weeks before tumor injection Compared to control group, mice fed with UroA diet as previously described (FIG. 1b) developed smaller tumors (FIG. 3). While mice fed with DMSO diet showed an average tumor size of 180 mm³ Urolithin A fed mice showed an average tumor size of 70 mm³.

Tumor engraftment, diet treatment and re-challenge experiments were as described initially in Example 1.

### Example 3

### Effect on Adaptive Immune Response

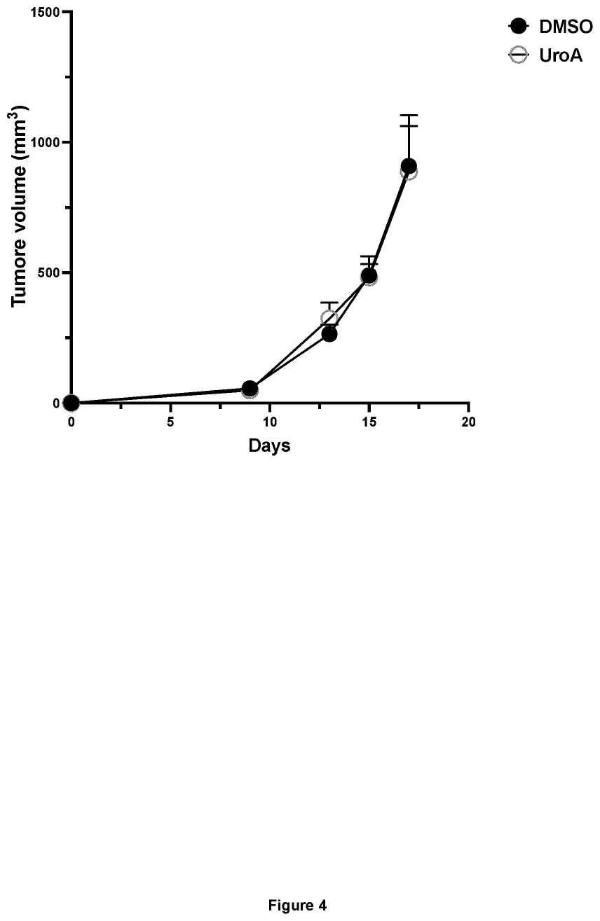

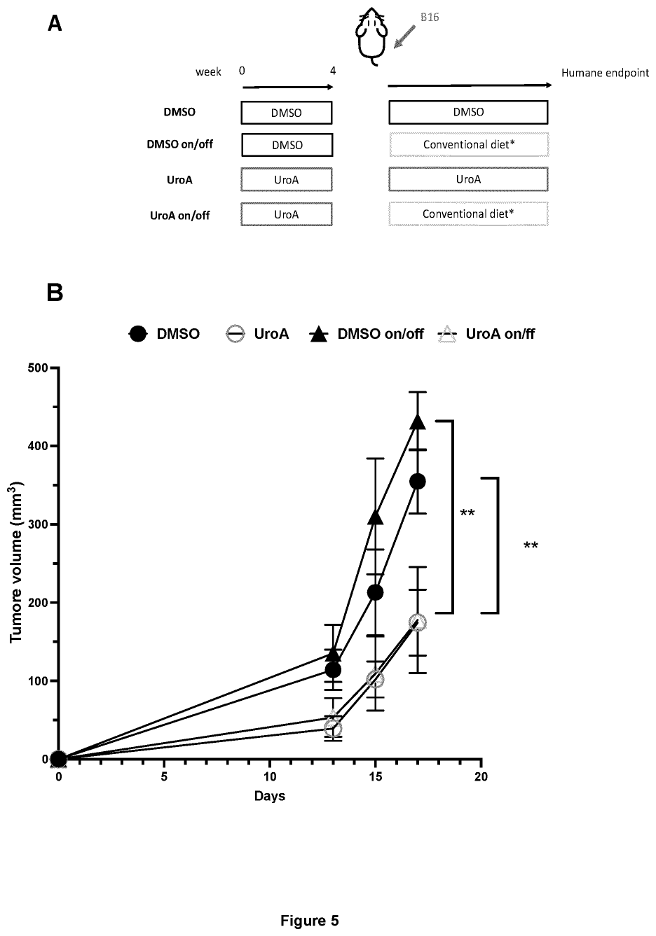

To validate that the Urolithin A supplementation enhances the adaptive immune response 5×10⁵ MC38 cells were subcutaneously injected in the right flank of the immunodeficient mice NSG (NOD.Cg-Prkdc^{sc}^{ⁱ}^{d} II2rg^{tm1Wil}/SzJ) previously fed with Urolithin A enriched diet for 4 weeks. Tumor volumes were estimated by using the formula: tumor volume=(length×width×width)/2. 5 mice recede the DMSO diet and 5 mice received the Urolithin A enriched diet. Both the 5 mice receiving the DMSO diet and the 5 mice receiving the Urolithin A enriched diet developed tumors. The tumors grow with the same rate and at the last timepoint (day 18), DMSO control mice and Urolithin A fed mice showed comparable tumor size (1000 m³). As can be seen from FIG. 4 there is therefore no effect on tumor size in the immunodeficient mice due to administration of UroA as evidenced by an identical tumor growth rate.

### Example 4

### Preventive Effect on Tumor Growth by Boosting the Immune System

To test whether Urolithin A feeding would have a preventive effect by boosting the immune system inventors performed the following experiment. A less immunogenic tumor model B16 melanoma was chosen for this experiment. 10 mice received ad libitum DMSO diet (see above), and 10 mice received ad libitum Urolithin A enriched diet (see above) for 4 weeks. Then 5 mice from DMSO diet were placed in conventional diet (provided by Animal facility), this group was called DMSO on/off. The remaining 5 mice were kept in DMSO diet and named DMSO group. 5 mice from Urolithin diet were placed in conventional diet (provided by Animal facility), this group was called UroA on/off. The remaining 5 mice were kept in DMSO diet and named UroA group. After one week, 5×10⁵ B16 cells were subcutaneously injected in the right flank of B57BL/6 mice.

Tumor engraftment, diet treatment and re-challenge experiments were as described initially in Example 1.

While mice fed in DMSO group or in DMSO on/off group showed similar tumor volumes, which reached an average tumor size of 350 mm³ and 430 mm³ at day 17, mice in UroA group and Uroa on/off group showed a reduced tumor growth and reached an average tumor size of 200 mm³ at day 17. These results indicate that a Urolithin A diet for 4 weeks has a preventive effect and generates a stronger immune response against cancer cells. The effect was maintained upon Urolithin A diet withdrawal, suggesting a possible imprinting mechanism of urolithin A on the immune system.

### SUMMARY

Based on inventor's observation, Urolithin dietary supplementation is able to reduce tumor growth in two different tumor models (FIG. 1 and FIG. 3). Both these preclinical models are used to investigate the contribution of immune cells, especially adaptive immune cells, in tumor control and tumor regression events. Under experimental conditions, Urolithin A supplementation was thereby able to generate a strong immune response capable to a) impede cancer cells to growth (FIG. 1) and b) to generate an adequate and strong immunological memory against cancer cells upon re-exposure to cancer development (FIG. 2). The generation of immunological memory is the aim of all established cell-based immunotherapies (e.g. vaccine). In presence of an immunological memory the immune system can quickly respond to the re-exposure to a pathogen or the potential relapse of a tumor. The immunological memory is guaranteed by the generation of specific set of T cells. To demonstrate that urolithin A supplementation boosted T cells function, inventors performed the same set of experiment in an immunodeficient host that lacks adaptive immune cells including T cells (FIG. 4). Under this experimental condition, Urolithin A supplementation did not affect tumor growth indicating a) Urolithin A does not affect cancer cell growth in the immunodeficient model, b) in absence of adaptive immune cells Urolithin A supplementation does not reduce tumor growth. These results indicate that Urolithin A is effective boosting the adaptive immune response against cancer cells under the experimental conditions. Use of Urolithin A is therefore considered to be particularly effective when used in the remission phase of a preceding cancer therapy.

All publications mentioned in the above specification are herein incorporated by reference. Various modifications and variations of the disclosed compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for use in reducing the risk of the recurrence of cancer or tumor formation in a patient in need thereof in the remission phase of a preceding cancer therapy in said patient comprising administering a composition comprising a therapeutically effective amount of a Urolithin or a precursor thereof to a patient in need of same.

2. The method according to claim 1, wherein the patient is a human or non-human mammal.

3. The method according to claim 1, wherein the patient is an immune-compromised patient.

4. The method according to claim 1, wherein the Urolithin or a precursor thereof is selected from the group consisting of a urolithin, ellagic acid, and an ellagitannin, more preferably is selected from Urolithin A, Urolithin B, Urolithin C or Urolithin D, and a combination thereof.

5. The method according to claim 1, wherein the urolithin is urolithin A, or a combination of Urolithin A with at least one of Urolithin B, Urolithin C or Urolithin D.

6. The method according to claim 1, wherein the urolithin is urolithin A.

7. The method according to claim 1, wherein the urolithin is administered in an amount of about 500 to 2000 mg/day.

8. The method according to claim 1, wherein the administration of the composition comprising a Urolithin starts at day 0 of the remission phase or at a later stage.

9. The method according to claim 1, wherein the cancer is a solid tumor.

10. The method according to claim 1, wherein the preceding cancer therapy is selected from the group consisting of a cancer surgery, a radiation therapy, a chemotherapy, a targeted cancer therapy, an immunotherapy, a hormonal therapy, and a phytochemical based cancer therapy.

11. The composition method according to claim 1, wherein the preceding cancer therapy involves use of a Urolithin.

12. The method according to claim 1, wherein the composition is administered orally.

13. The composition method according to claim 1, wherein the composition is in the form of a pharmaceutical or nutritional composition.

14. The method of claim 13, wherein the composition comprises an extract selected from the group consisting of pomegranate extract, a tamarind extract and a mumijo extract, that provides at least a portion of the at least one urolithin or precursor thereof.

15. The method of claim 14, wherein the composition comprises the Urolithin in an isolated form.

16. The composition method according to claim 1, wherein the composition furthermore comprises a NAD+ precursor and/or a Vitamin B, wherein the NAD+ precursor is selected from the group consisting Nicotinic Acid, Nicotinamide, Nicotinamide Riboside (NR), Reduced Nicotinamide riboside (NRH), Nicotinamide Mononucleotide (NMN), Nicotinic acid mononucleotide, Nicotinic acid riboside, and mixtures thereof.

17. The method according to claim 1, wherein the composition furthermore comprises Nicotinamide Riboside and Vitamin B12.

18. A method of reducing the reducing the risk of the recurrence of cancer or tumor formation in a patient in need thereof in the remission phase after a preceding cancer therapy in said patient, wherein the method comprises the following steps:
a) Providing a composition comprising a therapeutically effective amount of a Urolithin or a precursor thereof;
b) Administering the composition comprising the therapeutically effective amount of a Urolithin or a precursor thereof to the patient in need thereof, wherein the patient is in the remission phase after a preceding cancer therapy; and
c) Monitoring the patient regularly on the potential recurrence of the cancer treated in the preceding cancer therapy.

19-20. (canceled)
